(19) United States
(12) Patent Application Publication
(10) Pub. No.: US 2010/0240118 A1
(43) Pub. Date: Sep. 23, 2010

(54) COVER REMOVABLE DEVICE FOR CULTURE VESSEL AND CULTURE APPARATUS

(75) Inventor: Ryuji KOSHIBA, Yokohama-Shi (JP)

Correspondence Address: OLIFF & BERRIDGE, PLC, P.O. BOX 320850, ALEXANDRIA, VA, 22320-4850, US

(73) Assignee: NIKON CORPORATION, Tokyo (JP)

(21) Appl. No.: 12/748,815

(22) Filed: Mar. 29, 2010

Related U.S. Application Data
(63) Continuation of application No. PCT/JP2008/002919, filed on Oct. 15, 2008.

(30) Foreign Application Priority Data
Oct. 17, 2007 (JP) 2007-270190

(51) Int. Cl. C12M 1/36

(52) U.S. Cl. 435/286.2

(57) ABSTRACT

A lid removable device includes a first transferring mechanism transferring a culture vessel in a horizontal direction, a second transferring mechanism raising or lowering the culture vessel, a lid removable plate having an insertion region into which a vessel body of the culture vessel is inserted and a lid placing part on which a lid of the culture vessel is placed, and a transporting control unit controlling the first transferring mechanism so that the vessel body is inserted into the insertion region when the lid is removed from the culture vessel and controlling the second transferring mechanism so that the vessel body is separated from the lid, and controlling the first transferring mechanism so that the vessel body is positioned below the insertion region when the lid is attached to the culture vessel and controlling the second transferring mechanism so that the vessel body is moved toward the lid.

### CROSS REFERENCE TO RELATED APPLICATION

This application is a continuation application of International Application No. PCT/JP2008/002919, filed on Oct. 15, 2008, designating the U.S., in which the International Application claims a priority date of Oct. 17, 2007, based on prior filed Japanese Patent Application No. 2007-270190, the entire contents of which are incorporated herein by reference.

### BACKGROUND

1. Field

The present application relates to a lid removable device removing and attaching a lid of a culture vessel for culturing cells and the like and a culture apparatus.

2. Description of the Related Art

As a work for performing reagent addition, medium exchange, passage culture and the like for cells cultured in a culture vessel, first, a work for opening a lid of the culture vessel and accessing the inside of the culture vessel and then, closing the lid of the culture vessel is needed. This work, for example, can be accomplished by a method in which a culture vessel is automatically transported from a cabinet for storing the culture vessel (hereinafter referred to as a storage) and then, a lid of the culture vessel is opened/closed while the lid is adsorbed by an adsorption plate (see Japanese Unexamined Patent Application Publication No. 2004-305148).

### BRIEF DESCRIPTION OF THE DRAWINGS

FIG. 1 is an explanatory diagram illustrating an outline configuration of a cell culture observation device.

FIG. 2 is an explanatory diagram illustrating an outline configuration of a culture observation area of the cell culture observation device.

FIG. 3 is an exploded diagram illustrating a configuration of a lid removable unit.

FIG. 4 is an exploded diagram illustrating an example of a configuration of a culture vessel.

FIG. 5 is a perspective view illustrating a state when the culture vessel is moved toward the lid removable unit.

FIG. 6 is a perspective view illustrating a state in which the culture vessel is inserted into the lid removable unit.

FIG. 7 is a perspective view illustrating a state in which a lid of the culture vessel is removed by the lid removable unit.

FIG. 8 is a perspective view illustrating an embodiment in which a plurality of lid removable units are disposed in a storage.

### DETAILED DESCRIPTION OF THE EMBODIMENT

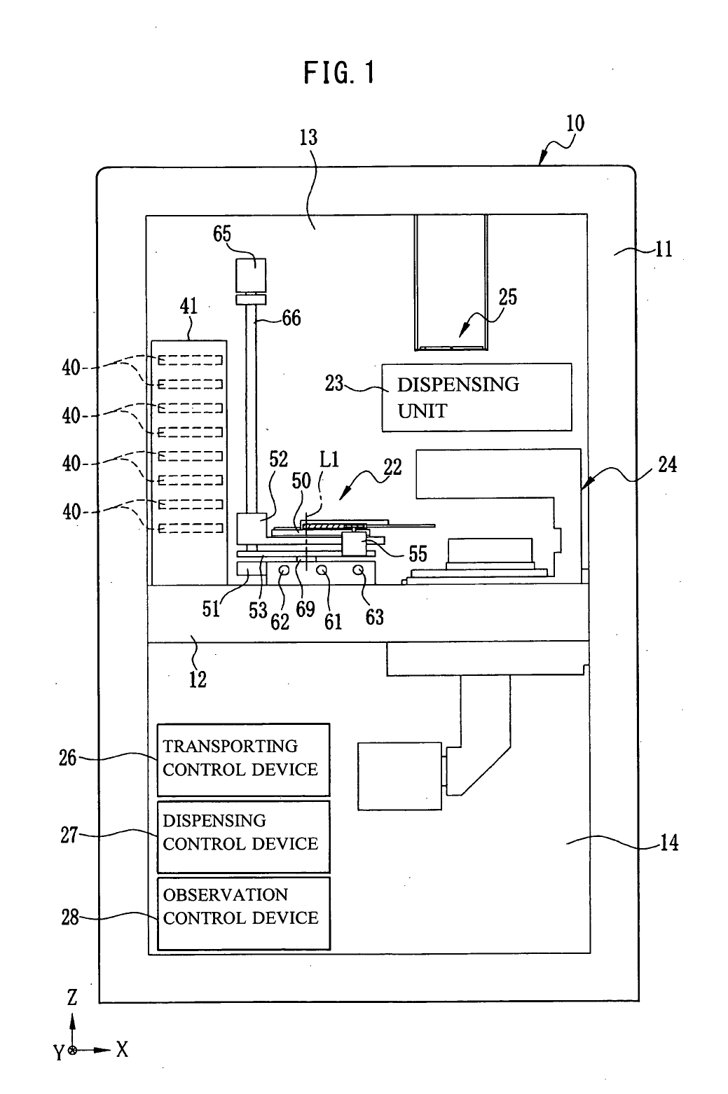

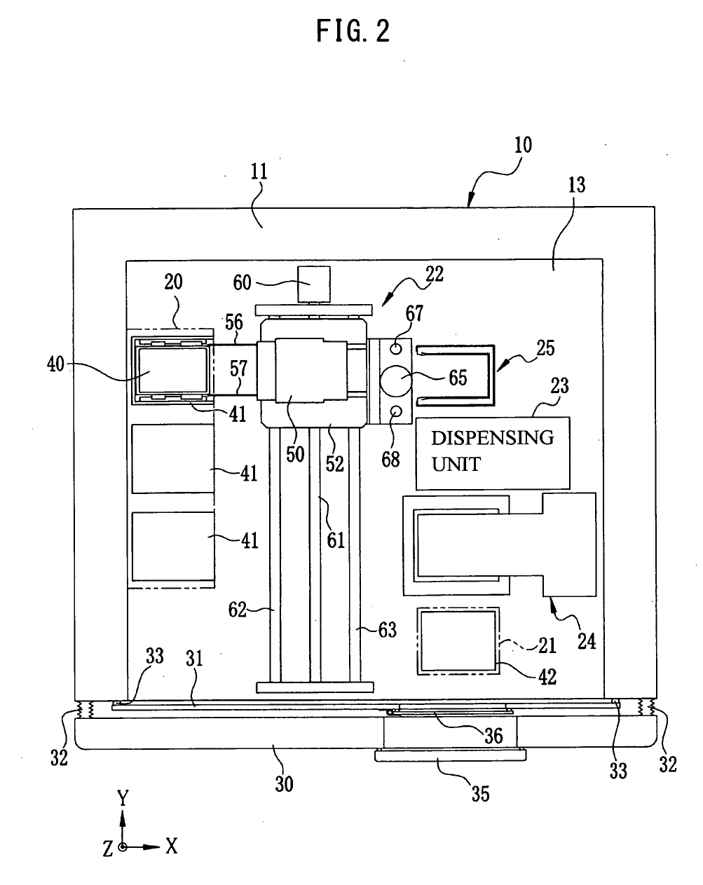

FIGS. 1 and 2 show an example of a configuration of a cell culture observation device 10. The cell culture observation device 10 is divided into two areas, that is, upper and lower areas 13 and 14 by providing a base plate 12 inside a housing 11. In the following, the area 13 above the base plate 12 in the area inside the housing 11 is referred to as the culture observation area 13 for explanation.

In the culture observation area 13, a transporting unit 22, a dispensing unit 23, an observation unit 24, and a lid removable unit 25 are provided in addition to a storage installation part 20 and a carrier installation part 21. In the meantime, in the area inside the housing 11, in the area 14 below the base plate 12, a transporting control device 26 controlling the transporting unit 22, a dispensing control device 27 controlling the dispensing unit 23, and an observation control device 28 controlling the observation unit 24 are disposed. The observation unit 24 is disposed across the two areas 13 and 14, and a packing (not shown) for making the culture observation area 13 airtight or preventing inflow of heat into the culture observation area 13 or outflow of heat from the culture observation area 13 is disposed between the observation unit 24 and the base plate 12.

At an upper part on the front face of this housing 11, an outer door 30 and an inner door 31 are attached pivotally. Packings 32 and 33 are disposed on peripheral edge parts on back faces of the outer door 30 and the inner door 31, respectively, and make the culture observation area 13 airtight by shutting off a gap generated between the respective doors 30 and 31 and the culture observation area 13 when the respective doors are closed. In addition, they prevent inflow of heat from outside of the cell culture observation device 10 into the culture observation area 13 and outflow of heat from inside the culture observation area 13 to the outside of the cell culture observation device 10. The culture observation area 13 is maintained under environmental conditions that a temperature is 37° C., moisture is 90%, and CO₂ concentration is 5%.

In the outer door 30, a carrying-in door 35 is disposed. This carrying-in door 35 is opened when a carrier 42 containing a culture vessel 40, which will be described later, is carried into the carrier installation part 21 or the carrier 42 installed in the carrier installation part 21 is carried out. When the carrying-in door 35 is opened, a small door 36 disposed in the inner door 31 is exposed. The inner door 31 and the small door 36 are formed by glass or a transparent synthetic resin and prevent the environmental conditions inside from being rapidly changed even when the outer door 30 is opened. The small door 36 disposed in the inner door 31 is opened together with the carrying-in door 18 when the carrier 42 containing the culture vessel 40 is carried into the carrier installation part 21 or when the carrier 42 is carried out of the carrier installation part 21.

As described above, inside the housing 11, the transporting unit 22, the dispensing unit 23, the observation unit 24, and the lid removable unit 25 are disposed in addition to the storage installation part 20 and the carrier installation part 21. In the storage installation part 20, three cabinets, for example, referred to as storages (hereinafter referred to as storage 41) capable of containing eight culture vessels 40 vertically, for example, are disposed along an inner wall face of the housing 11 (along the Y-axis direction). In the carrier installation part 21, the carrier 42 capable of stacking and containing three culture vessels 40, for example, is installed. This carrier 42 contains the culture vessel 40 containing cultured cells, the culture vessel 40 for which cells have been observed and the like.

The transporting unit 22 transports the culture vessel 40 contained in the carrier 42 to the storage 41 and transports the culture vessel 40 contained in the storage 41 to the dispensing unit 23, the observation unit 24 or the carrier 42. That is, this transporting unit 22 can transport the culture vessel 40 in the X-axis direction, the Y-axis direction, and the Z-axis direction.

This transporting unit 22 includes an X-stage 50, a Y-stage 51, a Z-stage 52, and a rotating stage 53. The X-stage 50 is attached to the Z-stage 52. This X-stage 50 moves in the X-axis direction based on driving of a motor 55. In addition, the X-stage 50 is telescopic in the X-axis direction. At a distal end of the X-stage 50, supporting arms 56 and 57 are disposed with a predetermined interval. The supporting arm 56 holds holding pieces 103 and 104 of a holder 100, which will be described later, while the supporting arm 57 holds holding pieces 105 and 106 of the holder, respectively.

The Y-stage 51 moves in the Y-axis direction by rotation of a driving screw shaft 61 fixed to a driving shaft of a motor 60. Reference numerals 62 and 63 denote guide shafts and regulate a moving direction of the Y-stage 51.

The Z-stage 52 is attached to the Y-stage 51 through the rotating stage 53. The Z-stage 52 moves in the Z-axis direction by rotation of a driving screw shaft 66 fixed to a driving shaft of a motor 65. Reference numerals 67 and 68 denote guide shafts and regulate a moving direction of the Z-stage 52.

The rotating stage 53 is attached to the Y-stage 51 through a driving shaft 69. To this driving shaft 69, a motor (not shown) is connected directly or indirectly, and by means of driving of the motor, the rotating stage 53 is rotated with the driving shaft 69 as a shaft, that is, around L1. According to this rotation, the X-stage 50 and the Z-stage 52 are also rotated.

The dispensing unit 23 is a unit that adds a solution such as reagent to each well 97 of the culture vessel 40 from which a lid 96 has been removed by the lid removable unit 25. The observation unit 24 is a unit for observation of cells inside each well 97 of the culture vessel 40, and this type of the observation unit 24 includes an existing microscope, for example.

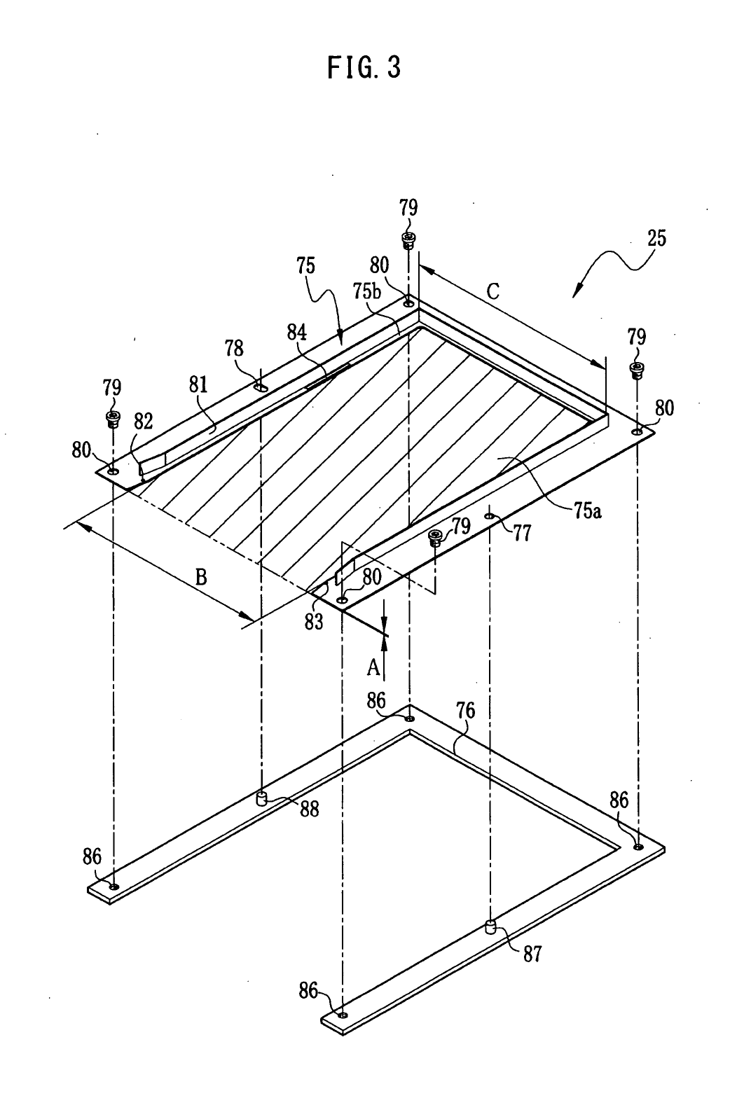

As shown in FIG. 3, the lid removable unit 25 includes a lid removable plate 75 and a fixing plate 76 that fixes the lid removable plate 75 to the inside of the housing 11. This. lid removable plate 75 is positioned to the fixing plate 76 by positioning pins 87 and 88 of the fixing plate 76 inserted through openings 77 and 78, respectively, and fixed to the fixing plate 76 by a screw 79, for example, while this state is maintained. Reference numeral 80 denotes an opening through which the screw 79 is inserted.

The lid removable plate 75 is made up of plate-shaped members, and the center part in its transverse direction is configured substantially in the U-shape from one end side to the other end side cut out in a rectangular shape in the longitudinal direction. To this rectangular cut-out portion (an insertion region indicated by hatching in FIG. 3) 75a, a peripheral wall 98 of a vessel body 95 of the culture vessel 40 is inserted. In the following this rectangular cut-out portion is described as the insertion region 75a. The vicinity around an inlet of the insertion region 75a is tapered so that the peripheral wall 98 of the vessel body 95 of the culture vessel 40 can be reliably inserted.

This lid removable plate 75 is formed such that its thickness A is smaller than a width D of a gap 99 between the vessel body 95 of the culture vessel 40 and the lid 96. The thickness A of the lid removable plate 75 is preferably set to approximately ⅓ of the width D of the gap 99. A gap B at a cut-out spot is formed narrower than a width F in the transverse direction of the lid 96 of the culture vessel 40 and wider than a width G in the transverse direction of the peripheral wall 98 formed in the vessel body 95.

On a top face of a lid placing part 75b of the lid removable plate 75, a positioning piece 81 substantially in the U-shape is formed in compliance with the shape of the lid removable plate 75. A width C of this positioning piece 81 is formed wider than the width F in the transverse direction of the lid 96, and the lid 96 is inserted into a region surrounded by the positioning piece 81 when the culture vessel 40 is moved toward the lid removable unit 25. The both end portions of the positioning piece 81 are bent in opposing directions so that the lid 96 to be inserted can easily enter the region surrounded by the positioning piece 81.

On the top face of the lid removable plate 75 and in the vicinity of the both end portions of the positioning piece 81, projections 82 and 83 that prevent drop of the lid 96 placed on the lid placing part 75b are disposed. Height of these projections 82 and 83 is preferably approximately ⅙ of the width D of the gap 99.

In the lid removable plate 75, a lid detection sensor 84 that detects presence of the lid 96 is disposed in the region inside the positioning piece 81. As the lid detection sensor 84, an optical sensor or the like as well as a contact sensor that detects contact can be used. It is necessary that the lid detection sensor 84 is disposed in a region inside the positioning piece 81, that is, the lid 96 is the lid placing part 75b of the lid removable plate 75. The lid detection sensor 80 can be installed in plural instead of singularly.

The fixing plate 76 is configured substantially in the U-shape similarly to the lid removable plate 75. This fixing plate 76 is held at a predetermined position of the housing 11 and holds the lid removable plate 75 at a predetermined position of the housing 11 by means of fixation of the lid removable plate 75 onto the top face of the fixing plate 76 with the screw 79, for example. Reference numeral 86 denotes a screw hole into which the screw 79 is screwed. At both end edges in the transverse direction of the fixing plate 76 and at the center part in the longitudinal direction, the positioning pins 87 and 88 inserted through the openings 77 and 78 of the lid removable plate 75 are disposed.

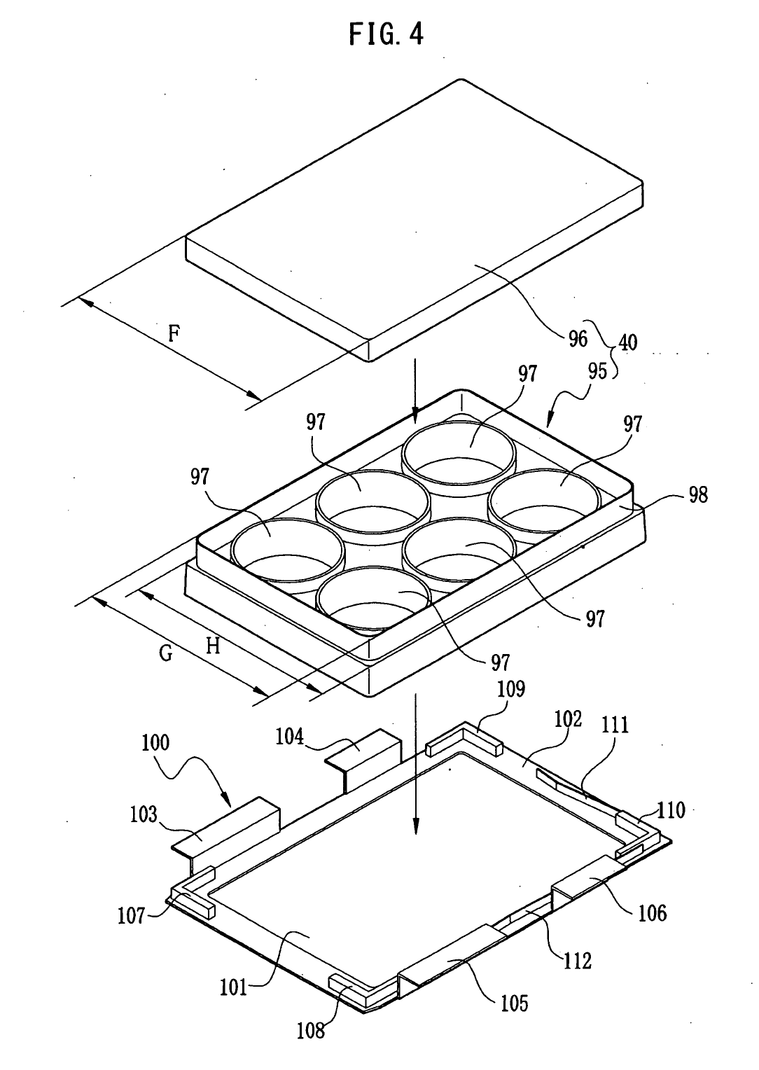

FIG. 4 is a perspective view illustrating a configuration of the culture vessel 40. The culture vessel 40 includes the vessel body 95 and the lid 96 removably attached to the upper face of the vessel body 95. The vessel body 95 and the lid 96 are made up of transparent glass or transparent synthetic resin, for example. The vessel body 95 is provided with six containers (wells) 97, for example, and cells to be cultured are contained in each of the wells 97.

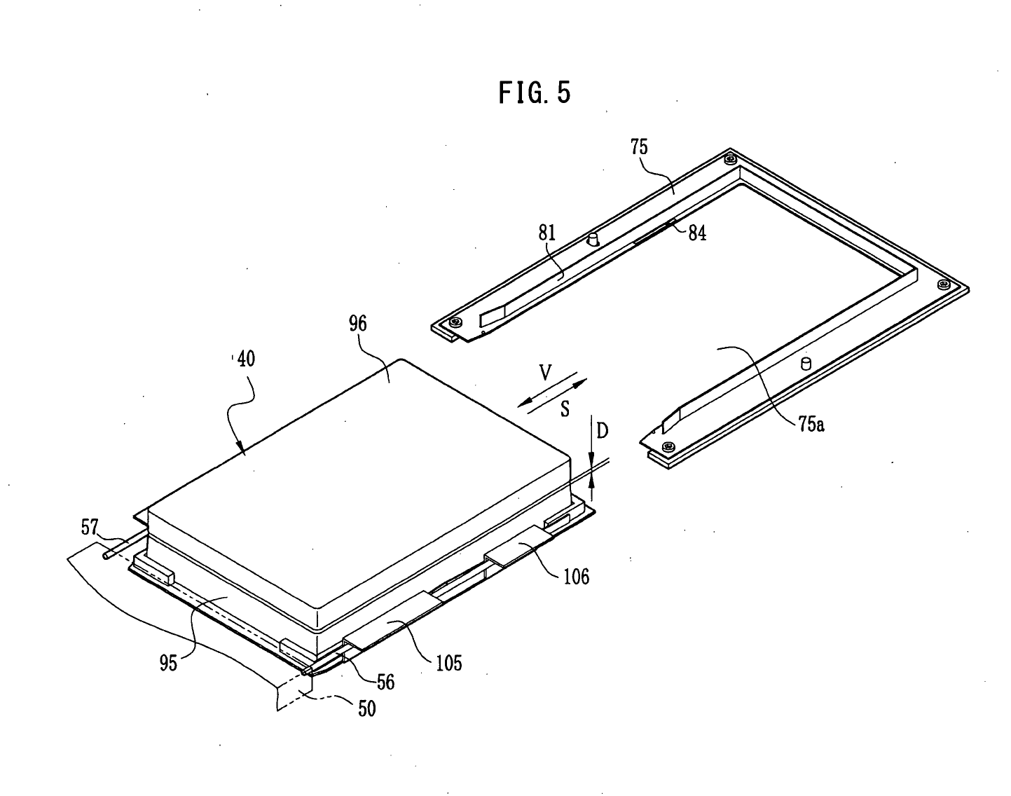

On the top face of the vessel body 95, the peripheral wall 98 is disposed so as to surround the wells 97 and also in compliance with the peripheral edge of the top face of the vessel body 95. With regard to this peripheral wall 98, its upper-end edge portion is contained inside the lid 96 when the lid 96 is to be attached so that the gap 99 with the width D is formed between the lid 96 and the vessel body 95 (See FIG. 5). The width H in the transverse direction on the top face of the vessel body 95 is formed so as to be equal to the width F in the transverse direction of the lid 96. As a result, when the lid 96 is attached to the vessel body 95, their side faces become substantially flush with each other.

While being held by the holder 100, the culture vessel 40 is contained inside the storage 41 and the carrier 42, respectively, or transported to each part. The holder 100 includes a base plate 102 in a rectangular plate shape in which an opening 101 is disposed at the center and holding pieces 103 to 106 disposed at both end portions in the transverse direction of the base plate 102. As mentioned above, since the culture vessel 40 is transported to each part in a state held by the holder 100, the opening 101 is disposed so that the cells contained in the culture vessel 40 held by the holder 100 can be seen through and observed when the culture vessel 40 is transported to the observation unit 25, for example. When the holder 100 is formed by a transparent synthetic resin or transparent glass or the like, the opening 101 does not necessarily have to be disposed.

At four corners on the upper face of the base plate 102, L-shaped positioning pieces 107 to 110 are disposed. These positioning pieces 107 to 110 position the vessel body 95 with respect to the base plate 102. In these positioning pieces 107 to 110, at both end portions of the positioning piece 110, plate springs 111 and 112 are attached, respectively. In these plate springs 111 and 112, the plate spring 111 extends toward the positioning piece 109, while the plate spring 112 extends toward the positioning piece 108. That is, the plate spring 111 brings the vessel body 95 into pressure contact with the positioning pieces 107 and 108 by pressing the center part in the transverse direction of the vessel body 95. The plate spring 112 brings the vessel body 95 into pressure contact with the positioning pieces 107 and 109 by pressing the center part in the longitudinal direction of the vessel body 95. As a result, when the lid 96 is to be removed, the vessel body 95 is prevented from departing from the holder 100.

The holding pieces 103 to 106 are disposed at the both end portions in the transverse direction of the base plate 102 and with a predetermined interval in the longitudinal direction of the base plate 102. The holding pieces 103 and 104 are supported by an arm portion 57 when they transport the culture vessel 40, while the holding pieces 105 and 106 are supported by an arm portion 56 when they transport the culture vessel 40. The holding pieces 103 to 106 are supported by holding pieces (not shown) of the storage 41 or the carrier 42 when the culture vessel 40 is contained in the storage 41 or the carrier 42.

Subsequently, a flow of attaching/removing the lid 96 of the culture vessel 40 using the lid removable unit 25 will be described. When a reagent is to be poured into the well 97 in the culture vessel 40, the transporting unit 22 is driven so as to insert the supporting arms 56 and 57 below the holder 100 of the applicable culture vessel 40. After that, the Z-stage 52 is raised by a predetermined amount. During rise of the Z-stage 52, the holder 100 is supported by the supporting arms 56 and 57. After that, by moving the X-stage 50, the holder 100 can be pulled out of the inside of the storage 41. After that, the rotating stage 53 is rotated by 180° around the driving shaft 69. After the rotation of the rotating stage 53, the culture vessel 40 is moved so that the peripheral wall 98 of the vessel body 95 in the culture vessel 40 enters the insertion region 75a of the lid removable plate 75. By means of this movement, in the Z-axis direction, the lid removable plate 75 is positioned within a range of the width D of the gap 99 generated between the vessel body 95 of the culture vessel 40 and the lid 96, and the projections 82 and 83 are positioned below the lower face of the lid 96.

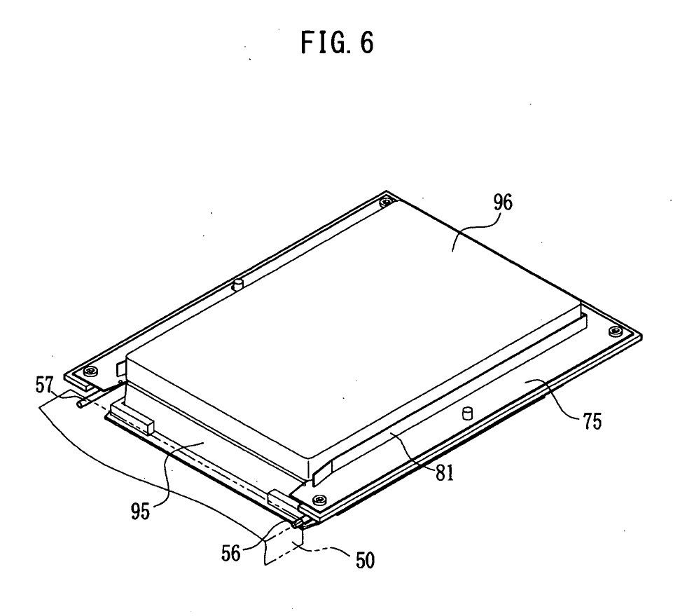

As shown in FIG. 5, the culture vessel 40 is moved to the lid removable unit 25 on the X-stage 50 in an S direction. When the X-stage 50 is moved in the S direction, the peripheral wall 98 of the vessel body 95 is inserted in the insertion region 75a of the lid removable plate 75. At this time, an edge of the lid placing part 75b of the lid removable plate 75 enters the gap 99 formed between the vessel body 95 of the culture vessel 40 and the lid 96 (See FIG. 6).

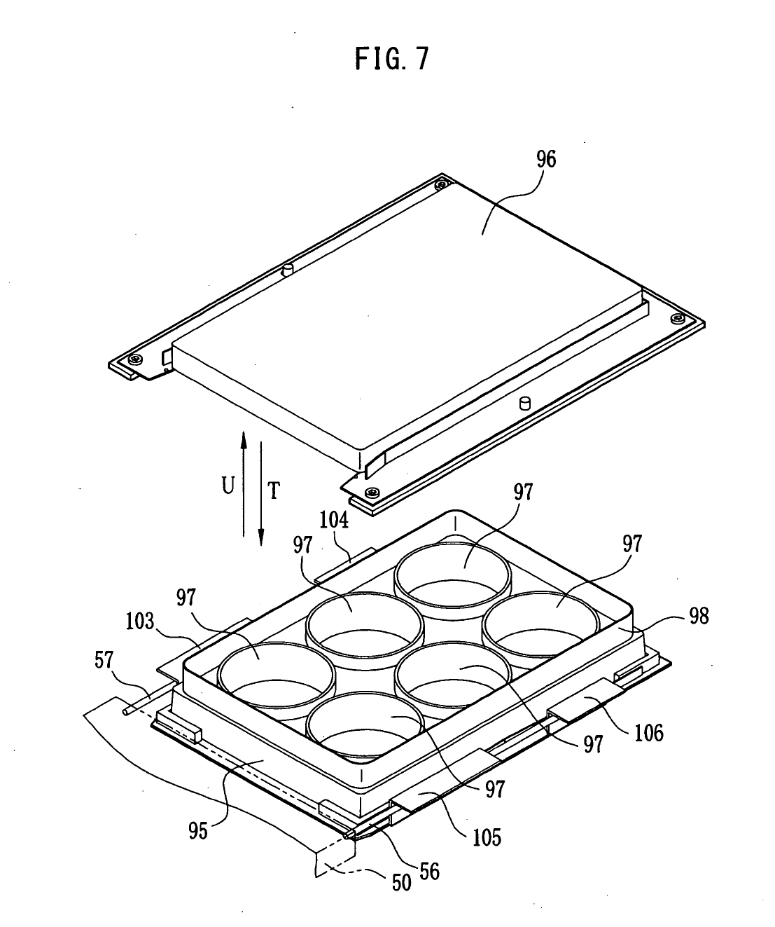

After that, by lowering the Z-stage 52 of the transporting unit 22, the vessel body 95 held by the holder 100 is moved in a T direction, and the lid 96 is brought into contact with the lid placing part 75b of the lid removable plate 75 and its movement in the T direction is regulated. As a result, as shown in FIG. 7, the lid 96 is placed on the lid placing part 75b of the lid removable plate 75, and the lid 96 is separated from the vessel body 95. Since the positioning piece 81 is disposed on the top face of the lid removable plate 75, during the course in which the lid 96 is separated from the vessel body 95, displacement of the lid 96 is prevented. When the culture vessel 40 is lowered, when the lid 96 is placed on the lid removable plate 75, the lid detection sensor 84 detects the lid 96, and wrong entry of another culture vessel 40 into the lid removable plate 75 in order to open the lid 96 of the different culture vessel 40 is prevented.

On the other hand, since the culture vessel 40 is pressed by the plate springs 111 and 112 disposed in the holder 100, while moving in the T direction, the vessel body 95 is prevented from departing from the holder 100.

The vessel body 95 lowered by the transporting unit 22 is separated from the lid 96 and then, transported to the dispensing unit 23, where a reagent is added to each well 97. After that, the vessel body 95 is transported from the dispensing unit 23 to below the insertion region 75a of the lid removable plate 75.

After this transportation, the Z-stage 52 is raised. The vessel body 95 is moved in a U direction by the rise of the Z-stage 52, and the peripheral wall 98 is inserted into the insertion region 75a of the lid removable plate 75. During this insertion, the upper-end edge portion of the peripheral wall 98 is contained inside the lid 96 placed on the lid placing part 75b. When the upper-end edge portion of the peripheral wall 98 is contained in the lid 96, the upper face of the peripheral wall 98 of the vessel body 95 is brought into contact with an inner face of the lid 96, and the lid 96 is raised by the vessel body 95. By the rise of the lid 96, the detection of the lid 96 by the lid detection sensor 84 is finished. In response to that, the Z-stage 52 is raised till the lower face of the lid 96 is positioned above the projections 82 and 83. As a result, the lid 96 is attached to the vessel body 95.

After the rise of the Z-stage 52 is stopped, the X-stage 50 is moved in a V direction, and the culture vessel 40 is taken out of the lid removable unit 25. When the cells immediately after addition of a reagent are to be observed, for example, the culture vessel 40 is transported toward the observation unit 24. On the other hand, when the cells immediately after the addition of the reagent are not to be observed, the culture vessel 40 is contained at a predetermined position in the storage 41.

As mentioned above, when the culture vessel 40 is transported by driving the transporting unit 22, the lid 96 of the culture vessel 40 can be attached/removed, and there is no need to newly provide a mechanism which attaches/removes the lid 96. In addition, since it is only necessary to provide the lid removable plate 75 inside the culture observation area 13, there is no need to provide a mechanism which adsorbs the lid 96, for example.

In this embodiment, the shape of the lid removable plate is substantially in the

U-shape, but not limited to that, and the lid removable plate may be made up of two rectangular plates arranged with a predetermined interval. In this case, it is only necessary that the predetermined interval is narrower than the width in the transverse direction of the lid and wider than the width in the transverse direction of the peripheral wall of the vessel body.

In this embodiment, the culture vessel in which a gap is formed between the lid and the vessel body is used so that the lid removable plate is inserted into the gap, but not limited to that, and a culture vessel in which the side face of the lid protrudes from the side face of the vessel body when the lid is attached to the vessel body may be used, for example. In this case, by inserting the vessel body into the insertion region of the lid removable plate below the lid, the lid is brought into contact with the lid removable plate when the culture vessel is lowered, and the lid can be removed from the vessel body.

In this embodiment, the shape or size of the culture vessel is not described in detail, but the culture vessels might be different in the shape or size depending on a manufacturer, for example. A plurality of lid removable plates may be prepared in advance in compliance with such culture vessels with different shapes or sizes so that the lid removable plate to be fixed to the fixing plate can be replaced according to the culture vessel to be used. Alternatively, when the lid removable plate can be replaced according to the culture vessel to be used, a work to replace the lid removable plate takes labor, and thus, it is possible to install the plurality of lid removable plates in advance in the culture observation area.

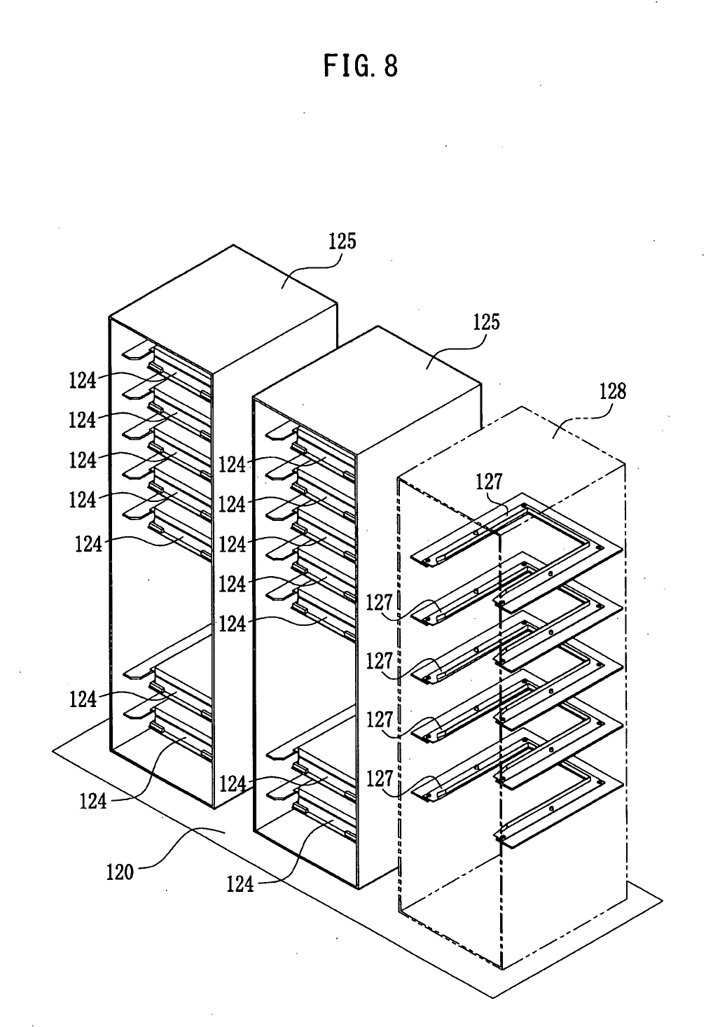

Moreover, a plurality of types of the lid removable plates may be disposed in one of a plurality of the storages installed in a storage installation part 120. As shown in FIG. 8, for example, a storage 125 containing the culture vessels 124 is installed in the storage installation part 120 and a storage 128 in which a plurality of types of the lid removable plates 127 are disposed vertically is installed. In this case, after the culture vessel 124 is taken out of the storage 125, the lid of the culture vessel 124 is removed or attached using the lid removable plate 127 matching the shape or size of the culture vessel 124.

When the lid removable plate is attached to the storage, in addition to a form in which the plurality of types of the lid removal plates are installed in one of the storages, there can be a form in which the lid removable plates are installed according to the shape or size of the culture vessel to be stored in each of the storages.

In this embodiment, the lid removable plate is disposed in the cell culture observation device, but the lid removable device of the present invention can be used in an automatic medium exchange device which automatically exchanges the cells to be cultured in the culture vessel or an automatic dispensing device which automatically adds a reagent into the culture vessel as long as it is a device which attaches/removes the lid of the culture vessel, for example.

The many features and advantages of the embodiment are apparent from the detailed specification and, thus, it is intended by the appended claims to cover all such features and advantages of the embodiment that fall within the true spirit and scope thereof. Further, since numerous modifications and changes will readily occur to those skilled in the art, it is not desired to limit the inventive embodiment to the exact construction and operation illustrated and described, and accordingly all suitable modifications and equivalents may be resorted to, falling within the scope thereof.

1. A lid removable device for a culture vessel including a vessel body in which a housing containing a sample is formed and a lid which covers an upper face and a part of a side face of the vessel body, comprising:
a first transferring mechanism which transfers the culture vessel in a horizontal direction;
a second transferring mechanism which raises or lowers the culture vessel;
a lid removable plate having an insertion region into which the vessel body of the culture vessel is inserted and a lid placing part on which the lid of the culture vessel is placed; and
a transporting control unit controlling the first transferring mechanism so that the vessel body is inserted into the insertion region of the lid removable plate when the lid is removed from the culture vessel and controlling the second transferring mechanism so that the vessel body is separated while the lid is placed on the lid placing part of the lid removable plate, and controlling the first transferring mechanism so that the vessel body of the culture vessel is positioned below the insertion region of the lid removable plate when the lid is attached to the culture vessel and controlling the second transferring mechanism so that the vessel body is moved toward the lid on the lid placing part of the lid removable plate.

2. The lid removable device for the culture vessel according to claim 1, wherein:
the lid removable plate is made up of a plate-shaped member having a thickness smaller than an interval of a gap generated between the lid of the culture vessel and the vessel body;
the lid placing part is formed on an upper face side of the plate-shaped member; and
the insertion region is formed by cutting out a part of the plate-shaped member so that an edge of the lid placing part enters the gap generated between the lid of the culture vessel and the vessel body when the vessel body of the culture vessel is inserted into the insertion region.

3. The lid removable device for the culture vessel according to claim 1, wherein
the lid placing part of the lid removable plate includes a positioning piece which positions the lid of the culture vessel inserted into the insertion region.

4. The lid removable device for the culture vessel according to claim 1, wherein
the lid removable plate includes a lid sensing unit which senses the lid being placed on.

5. The lid removable device for the culture vessel according to claim 1, wherein
the lid removable plate includes a dropout-preventing projection of the lid being placed on.

6. The lid removable device for the culture vessel according to claim 1, wherein
the lid removable plate is replaceable in accordance with a shape of the culture vessel.

7. A culture apparatus in which the lid removable device for the culture vessel according to claim 1 is used, comprising
a storage containing the culture vessel, wherein
the lid removable plate is placed in the storage.

8. A culture apparatus in which the lid removable device for the culture vessel according to claim 2 is used, comprising
a storage containing the culture vessel, wherein
the lid removable plate is placed in the storage.

9. A culture apparatus in which the lid removable device for the culture vessel according to claim 3 is used, comprising
a storage containing the culture vessel, wherein
the lid removable plate is placed in the storage.

10. A culture apparatus in which the lid removable device for the culture vessel according to claim 4 is used, comprising
a storage containing the culture vessel, wherein
the lid removable plate is placed in the storage.

11. A culture apparatus in which the lid removable device for the culture vessel according to claim 5 is used, comprising
a storage containing the culture vessel, wherein
the lid removable plate is placed in the storage.

12. A culture apparatus in which the lid removable device for the culture vessel according to claim 6 is used, comprising
a storage containing the culture vessel, wherein
the lid removable plate is placed in the storage.
